(19) United States
(12) Patent Application Publication
(10) Pub. No.: US 2015/0366449 A1
(43) Pub. Date: Dec. 24, 2015

(54) PATIENT EYE LEVEL DEVICE AND METHOD

(71) Applicant: NOVARTIS AG, BASEL (CH)

(72) Inventor: TAKAYUKI AKAHOSHI, TOKYO (JP)

(21) Appl. No.: 14/309,508

(22) Filed: Jun. 19, 2014

(51) Int. Cl. A61B 3/00; G01B 5/06; A61F 9/007

(52) U.S. Cl. CPC A61B 3/0083; A61F 9/00745; G01B 5/061

(57) ABSTRACT

A method of determining a vertical location of a patient's eye includes receiving in a surgical console a location of a surgical tray coupled to the surgical console and calculating a vertical distance relative to a point based on the location of the surgical tray.

### BACKGROUND

The present invention relates to surgical systems and more particularly to a method and device for establishing the height of a patient's eye during a surgical procedure.

The human eye functions to provide vision by transmitting light through a clear outer portion called the cornea, and focusing the image by way of a crystalline lens onto a retina. The quality of the focused image depends on many factors including the size and shape of the eye, and the transparency of the cornea and the lens. When age or disease causes the lens to become less transparent, vision deteriorates because of the diminished light which can be transmitted to the retina. This deficiency in the lens of the eye is medically known as a cataract. An accepted treatment for this condition is surgical removal of the lens and replacement of the lens function by an artificial intraocular lens (IOL).

In the United States, the majority of cataractous lenses are removed by a surgical technique called phacoemulsification. A typical surgical instrument suitable for phacoemulsification procedures on cataractous lenses includes an ultrasonically driven phacoemulsification hand piece, an attached hollow cutting needle surrounded by an irrigating sleeve, and an electronic control console. The hand piece is attached to the control console by an electric cable and flexible tubing. Through the electric cable, the console varies the power level transmitted by the hand piece to the attached cutting needle. The flexible tubing supplies irrigation fluid to the surgical site and draws aspiration fluid from the eye through the hand piece.

During a phacoemulsification procedure, the tip of the cutting needle and the end of the irrigation sleeve are inserted into the anterior segment of the eye through a small incision in the eye's outer tissue. The surgeon brings the tip of the cutting needle into contact with the lens of the eye, so that the vibrating tip fragments the lens. The resulting fragments are aspirated out of the eye through the interior bore of the cutting needle, along with irrigation fluid provided to the eye during the procedure, and into a waste reservoir.

Throughout the procedure, irrigating fluid is infused into the eye, passing between the irrigation sleeve and the cutting needle and exiting into the eye at the tip of the irrigation sleeve and/or from one or more ports or openings formed into the irrigation sleeve near its end. This irrigating fluid prevents the collapse of the eye during the removal of the emulsified lens. The irrigating fluid also protects the eye tissues from the heat generated by the vibrating of the ultrasonic cutting needle. Furthermore, the irrigating fluid suspends the fragments of the emulsified lens for aspiration from the eye.

Conventional systems employ fluid-filled bottles or bags hung from an intravenous (IV) pole as an irrigation fluid source. Irrigation flow rates, and corresponding fluid pressure at the eye, are regulated by controlling the height of the IV pole above the surgical site. For example, raising the IV pole results in a corresponding increase in head pressure and increase in fluid pressure at the eye, resulting in a corresponding increase in irrigation flow rate. Likewise, lowering the IV pole results in a corresponding decrease in pressure at the eye and corresponding irrigation flow rate to the eye. Therefore, in order to maintain proper pressure in the eye during surgery, it is desirable to know the vertical distance between the bottle or bag and the eye.

### SUMMARY OF THE INVENTION

The present disclosure describes several examples of the invention. In one example, a method of determining a vertical location of a patient's eye includes: receiving in a surgical console a location of a surgical tray coupled to the surgical console; and calculating a vertical distance relative to a point based on the location of the surgical tray.

The point may be a connection point at which a tray arm is coupled to the surgical console, ground level, at an irrigation source coupled to the surgical console, or at an aspiration pump located at the console. In some cases, the vertical distance is a patient eye level. The vertical distance may correspond to a pressure at the patient's eye. In such a case, the pressure is used to control a parameter of the surgical console.

The method may also comprise receiving an angle of a tray arm coupled to the surgical tray, the angle of the tray arm relative to a line containing a connection point at the surgical console and using the angle to calculate the vertical distance.

The method may also comprise receiving from a position sensor a vertical location of the surgical tray, receiving an input on a touch screen indicating that the surgical tray has been placed at the vertical location of the patient's eye, providing an indication to a user of the surgical console to place the surgical tray at the vertical location of the patient's eye, and/or correlating the vertical distance to the vertical location of the patient's eye.

It is to be understood that both the foregoing general description and the following detailed description are exemplary and explanatory only and are intended to provide further explanation of the invention as claimed. The following description, as well as the practice of the invention, set forth and suggest additional advantages and purposes of the invention.

### BRIEF DESCRIPTION OF THE FIGURES

The accompanying drawings, which are incorporated in and constitute a part of this specification, illustrate several embodiments of the invention and together with the description, serve to explain the principles of the invention.

FIG. 1 illustrates a patient eye level (PEL) relative to a surgical component.

FIG. 2 illustrates an exemplary phacoemulsification surgical console.

FIG. 3 is a representation of an exemplary PEL device and method.

FIG. 4 is an example of a tray arm system.

FIG. 5 is an example of a tray arm system.

FIG. 6 is an exemplary method of using a tray to determine patient eye level.

### DETAILED DESCRIPTION

Reference is now made in detail to the exemplary embodiments of the invention, examples of which are illustrated in the accompanying drawings. Wherever possible, the same reference numbers are used throughout the drawings to refer to the same or like parts.

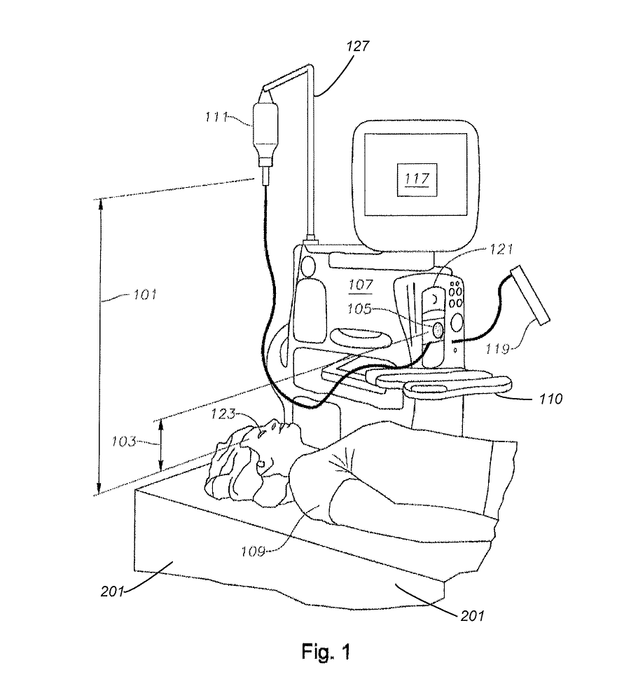

FIG. 1 illustrates a patient eye level (PEL) 101 relative to a surgical component (e.g., an irrigation bottle 111). The PEL 101 may be a vertical height between an outlet of the irrigation bottle 111 and a patient's eye 123 when the patient 109 is lying on a surgical table 201. As seen in FIG. 1, “vertical height” may include a perpendicular distance (e.g., in centimeters) between the patient's eye 123 and a line, parallel to the ground or floor, that intersects the outlet end of the irrigation bottle 111. While several embodiments presented herein describe the PEL as a vertical height between the patient's eye 123 and the irrigation bottle 111, it is to be understood that the PEL may also be a vertical height between the patient's eye 123 and a different surgical component of the surgical console 107 (e.g., a sensor 105 in which case the PEL is indicated by 103) or another reference point (e.g., the ground). In some embodiments, the PEL 101 may be used by the surgical console 107 in determining, for example, an aspiration or irrigation pressure at a patient's eye 123. For example, an aspiration pump 121 may provide aspiration to hand piece 119 through a fluid line coupling the hand piece 119 to the console 107. In some embodiments, the surgical console 107 may determine an approximate aspiration pressure at the patient's eye 123 (e.g., at the tip of the hand piece 119) using sensor readings at the aspiration sensor 105 (on that fluid line) in combination with the PEL. The PEL may also be used in determining an irrigation pressure. For example, irrigation pressure may increase with increasing PEL. The greater a bottle height (relative to a patient's eye 123), the greater the pressure of irrigation fluid entering the patient's eye 123 from the bottle 111. The surgical console 107 may use the pressure information to control a source of irrigation or aspiration (e.g., control an aspiration pump speed, an irrigation bottle height 101, etc).

In FIG. 1, a patient 109 is located on a surgical table 201. A console 107 includes a sensor 105, an aspiration pump 121, and a display 117. The sensor 105 may be a pressure or flow sensor. When sensor 105 is a pressure sensor, it typically measures the pressure of the irrigation fluid from irrigation bottle 111 or pressure in the aspiration line from hand piece 119. In some cases, two pressure sensors are present—one measuring the irrigation pressure and the other measuring the aspiration pressure. In this example, an IV pole 127 holds an irrigation bottle 111 that contains irrigation fluid used during surgery. A tray 110 is coupled to console 107. Tray 110 is located in the sterile field during surgery and holds various instruments. A hand piece 119 is coupled to console 107 typically via tubing through which irrigation fluid flows during surgery. In cataract surgery, hand piece 119 is typically a phacoemulsification hand piece. In this case, hand piece 119 is coupled to an irrigation line that provides irrigation fluid to the hand piece 119 from irrigation bottle 111. Hand piece 119 is also coupled to an aspiration line that in turn is coupled to an aspiration pump 121. The aspiration pump 121 draws fluid from the eye 123, through the hand piece, and to a drain bag. The hand piece 119 is used to perform surgery on the patient's eye 123.

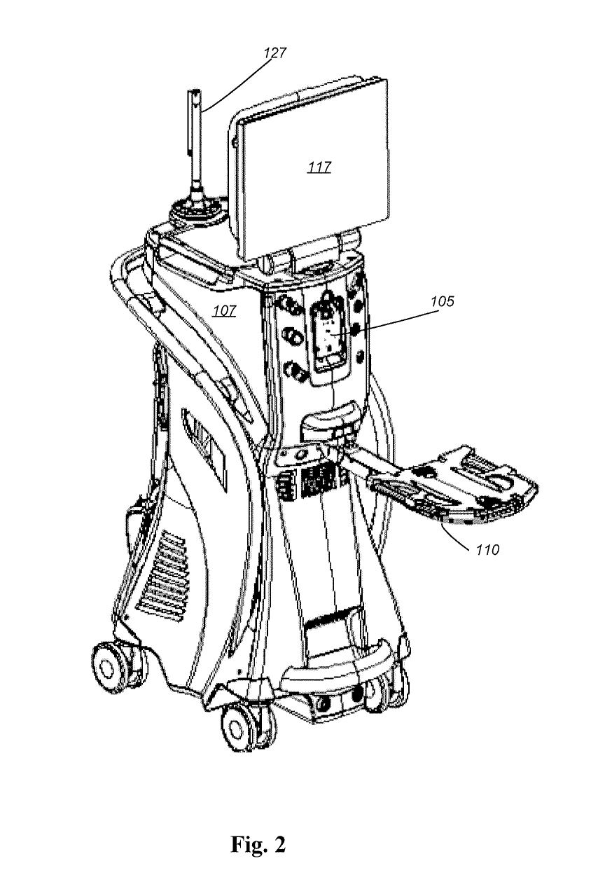

FIG. 2 illustrates an exemplary phacoemulsification surgical console 107. In FIG. 2, console 107 has a sensor 105, a surgical tray 110, a display 117, and an IV pole 127. During a surgical procedure, sensor 105 is located on surgical console 107 and typically measures a pressure in the irrigation or aspiration line coupled to the surgical hand piece. Surgical tray is fixed on one end to surgical console 107. Surgical tray 110 provides a work surface for use during surgery. An articulating arm couples surgical tray 110 to surgical console 107. This articulating arm allows surgical tray 110 to be adjusted to a suitable height above the floor. Display 117 is typically a touch screen display that provides information to and receives inputs from a user. In FIG. 2, IV pole 127 is shown in a folded configuration. IV pole 127 is typically adjustable and holds an irrigation fluid source such as a bottle or bag.

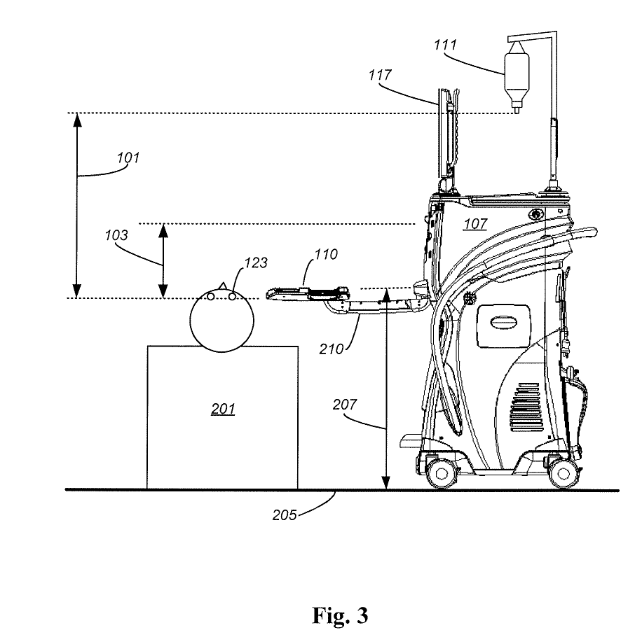

FIG. 3 is a representation of an exemplary PEL device and method. In FIG. 3, the position of the surgical tray 110 is used to determine PEL 101. Tray 110 is coupled to console 107 via an articulating tray arm 210. The height and position of tray 110 can be adjusted by moving tray 110. Typically, tray arm 210 is an articulating arm that can be locked into place so that tray 110 is stable. Tray arm 210 is also capable of being unlocked so that tray 110 can be freely moved to a suitable position. When tray 110 is used to determine PEL 101, a person unlocks tray arm 210 by grasping tray 110, moves tray 110 so that it is level with an eye 123. The user can then provide an input to the console 107, for example via display 117, indicating that the tray 110 is at the level of the patient's eye 123. In another example, the console 107 may request that that the person locate the tray 110 so that it is level with the eye 123 and then ask for confirmation of such location, for example via display 117.

In FIG. 3, the relative position of tray 110 with respect to console 107 can be determined since the dimensions of tray 110 and tray arm 210 are known and the positioning of tray arm 210 can be determined (as described in more detail later). In FIG. 3, tray 110 is located at the level of eye 123. In this case, tray is also located a distance 103 (one example of PEL) from a point on console 107 at which sensor is located. Tray 110 is also located a distance equal to PEL 101 from the head of irrigation bottle 111. In addition, the point at which tray arm 210 is connected to console 107 is a distance 207 from the floor 205.

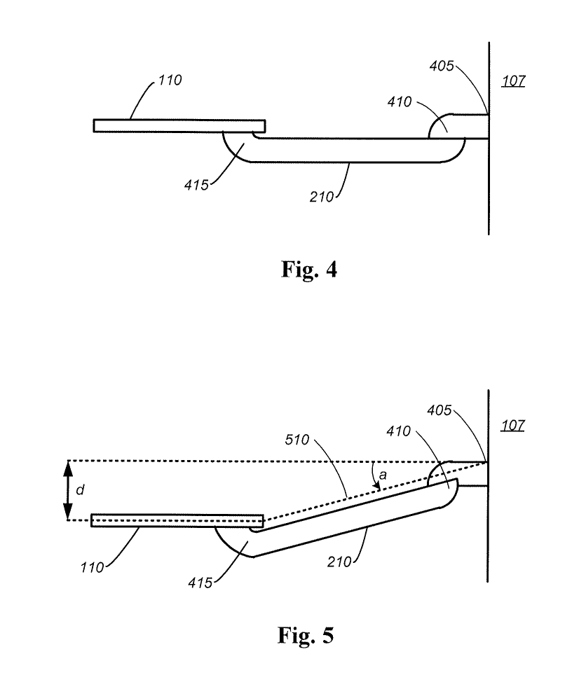

FIG. 4 is an example of a tray arm system. In FIG. 4, tray 110 is coupled to tray arm 210 by tray joint 415. Tray arm 210 is coupled to console 107 at connection point 405 by console joint 410. Console joint 410 and tray joint 415 both articulate such that tray 110 can be moved up and down as well as side to side. In this case, it is the up and down movement that can be used to determine the position of tray 110 relative to connection point 405.

FIG. 5 is an example of a tray arm system. In FIG. 5, tray 110 has been moved vertically downward with respect to connection point 405. For example, tray 110 may be moved downward in this manner so that it is level with a patient's eye. In this position, tray 110 is a distance “d” lower than connection point 405. Console joint 410 and tray joint 415 have both moved through an angle “a.” The distance “d” can be calculated based on the angle “a,” for example by: sin(a) x (length of dashed line 510). The height of the tray 110 above the ground can also be calculated since the distance between connection point 405 and the ground is known (i.e. connection point 405 is at a particular known location on the console). The PEL can also be calculated from the distance “d” and the height of irrigation bottle 110 above connection point 405. Other methods of calculating the vertical location of tray 110 may also be employed. For example, a vertical position sensor may be employed on tray 110, differential position sensors may be employed on tray 110 and at connection point 405, or angle sensors may be employed at console joint 410 and/or tray joint 415.

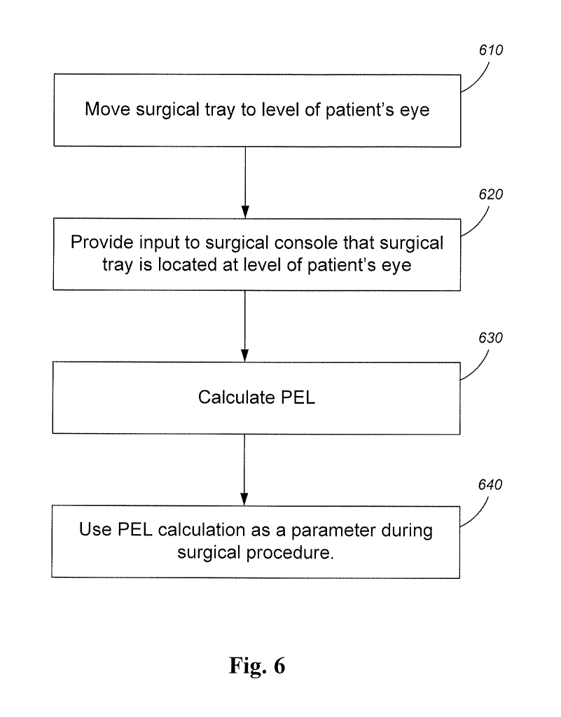

FIG. 6 is an exemplary method of using a tray to determine patient eye level (PEL). In 610, the surgical tray is moved so that it is level with a patient's eye. In 620, an input is provided to the surgical console that the surgical tray is located at the level of the patient's eye. In 630, the PEL is calculated. In 640, the PEL calculation is used as a parameter during a surgical procedure. Since the PEL represents the height of the irrigation source above the patient's eye, it also reflects the pressure of the irrigation fluid at the patient's eye. This pressure can be used to control the surgical system to maintain a particular pressure in the eye during surgery.

From the above, it may be appreciated that the present invention provides a method and device for measuring a patient's eye level. The present disclosure describes using a surgical tray to determine the location of a patient's eye relative to a point on the console, the ground level, or an irrigation source. The present invention is illustrated herein by example, and various modifications may be made by a person of ordinary skill in the art. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of determining a vertical location of a patient's eye, the method comprising:
receiving in a surgical console a location of a surgical tray coupled to the surgical console; and
calculating a vertical distance relative to a point based on the location of the surgical tray.

2. The method of claim 1 wherein the point is a connection point at which a tray arm is coupled to the surgical console.

3. The method of claim 1 wherein the point is at ground level.

4. The method of claim 1 wherein the point is at an irrigation source coupled to the surgical console.

5. The method of claim 1 wherein the point is at an aspiration pump located at the console.

6. The method of claim 1 wherein the vertical distance is a patient eye level.

7. The method of claim 1 wherein the vertical distance corresponds to a pressure at the patient's eye.

8. The method of claim 7 wherein the pressure is used to control a parameter of the surgical console.

9. The method of claim 1 wherein receiving in a surgical console a location of a surgical tray coupled to the surgical console further comprises:
receiving an angle of a tray arm coupled to the surgical tray, the angle of the tray arm relative to a line containing a connection point at the surgical console.

10. The method of claim 9 wherein calculating a vertical distance relative to a point based on the location of the surgical tray further comprises using the angle to calculate the vertical distance.

11. The method of claim 1 wherein receiving in a surgical console a location of a surgical tray coupled to the surgical console further comprises:
receiving from a position sensor a vertical location of the surgical tray.

12. The method of claim 1 wherein receiving in a surgical console a location of a surgical tray coupled to the surgical console further comprises:
receiving an input on a touch screen indicating that the surgical tray has been placed at the vertical location of the patient's eye.

13. The method of claim 1 further comprising:
providing an indication to a user of the surgical console to place the surgical tray at the vertical location of the patient's eye.

14. The method of claim 1 further comprising:
correlating the vertical distance to the vertical location of the patient's eye.
